(19) United States
(12) Patent Application Publication
(10) Pub. No.: US 2022/0409100 A1
(43) Pub. Date: Dec. 29, 2022

(54) REAL TIME FLUORESCENT DETECTION SYSTEMS FOR MEDICAL DEVICES

(71) Applicant: Acies Medical LLC, Saint Paul, MN (US)

(72) Inventors: Daniel Sipple, Saint Paul, MN (US); Brian L. Norling, Santa Barbara, CA (US)

(21) Appl. No.: 17/779,250

(22) PCT Filed: Dec. 4, 2020
(86) PCT No.: PCT/US2020/063181

§ 371 Date: May 24, 2022

Related U.S. Application Data
(60) Provisional application No. 62/954,404, filed on Dec. 27, 2019.

(51) Int. Cl. A61B 5/1459; A61B 5/00; A61B 5/1473; A61B 5/145

(52) U.S. Cl. CPC A61B 5/1459; A61B 5/6848; A61B 5/14735; A61B 5/14546; A61M 2025/0095

(57) ABSTRACT

A probe for real-time sensing of a target biomarker the includes a needle, a luminescent probe within the opening of the needle, a coating comprising a biomarker luminescent material in contact with biological tissue, and an ion-consuming coating within the needle and adjacent to the coating. The disclosed probe is useful for real-time sensing of blood during medical procedures. Additionally, a biomarker detection system is disclosed that includes a biomarker luminescent material at the tip of or inside of the tip of a needle and an optical coupler.

### CROSS-REFERENCE TO RELATED APPLICATIONS

This application claims the benefit of U.S. Provisional Patent Application Ser. No. 62/954,404, currently pending and titled, “REAL TIME FLUORESCENT DETECTION SYSTEMS FOR MEDICAL DEVICES”, which was filed Dec. 27, 2019, and is hereby incorporated by reference in its entirety.

### FIELD

The present disclosure relates to systems and methods for real time detection of biological substances and distinguishing between bodily tissues.

### BACKGROUND

Efforts to improve surgical outcomes and cost structure, particularly with spinal surgery, have led to increased use of minimally invasive procedures. These procedures often use image-guided modalities such as fluoroscopy, CT, nerve stimulators, and, more recently, Doppler ultrasound. While often involving less risk than surgery, minimally invasive spinal procedures, pain management procedures, nerve blocks, ultrasound guided interventions, biopsy, and percutaneous placement or open intra-operative placement continue to carry risks of ineffective outcome and iatrogenic injuries, such as infection, stroke, paralysis and death due to penetration of various structures including, but not limited to, organs, soft tissues, vascular structures, and neural tissue such as, catastrophically, the spinal cord. Injuries can occur regardless of practitioner experience because a surgical instrument must proceed through several layers of bodily tissues and fluids to reach the desired space in the spinal canal.

To illustrate, the intrathecal (or subarachnoid) space of the spinal region, where many medications are administered, houses nerve roots and cerebrospinal fluid (CSF) and lays between two of the three membranes that envelope the central nervous system. The outermost membrane of the central nervous system is the dura mater, the second is the arachnoid mater, and the third, and innermost membrane, is the pia mater. The intrathecal space is in between the arachnoid mater and the pia mater. To get to this area, a surgical instrument may need to first get through skin layers, fat layers, the interspinal ligament, the ligamentum flavum, the epidural space, the dura mater, the subdural space, and the intrathecal space. Additionally, in the case of a needle used to administer medication, the entire needle opening must be within the sub-arachnoid space. Because of the complexities involved in inserting a surgical instrument into the intrathecal space, penetration of the spinal cord and neural tissue is a known complication of minimally invasive spine procedures and spine surgery. Additionally, some procedures require the use of larger surgical instruments. For example, spinal cord stimulation, a form of minimally invasive spinal procedure wherein small wire leads can be inserted in the spinal epidural space, may require that a 14-gauge needle be introduced into the epidural space in order to thread the stimulator lead. Needles of this gauge can be technically more difficult to control, posing a higher risk of morbidity. Complications can include dural tear, spinal fluid leak, epidural vein rupture with subsequent hematoma, and direct penetration of the spinal cord or nerves with resultant paralysis. These and other high-risk situations, such as spinal interventions and radiofrequency ablation, can occur when a practitioner is unable to detect placement of the needle or surgical apparatus tip in critical anatomic structures.

At present, detection of such structures is operator dependent, wherein operators utilize tactile feel, contrast agents, anatomical landmark palpation and visualization under image-guided modalities. The safety of patients can rely upon the training and experience of the practitioner in tactile feel and interpretation of the imagery. Even though additional training and experience may help a practitioner, iatrogenic injury can occur independently of practitioner experience and skill because of anatomic variability, which can arise naturally or from repeat procedures in the form of scar tissue. Fellowship training in some procedures, such as radiofrequency ablation, may not be sufficiently rigorous to ensure competence; even with training, outcomes from the procedure can vary considerably. In the case of epidural injections and spinal surgery, variability in the thickness of the ligamentum flavum, width of the epidural space, dural ectasia, epidural lipomatosis, dural septum, and scar tissue all can add challenges to traditional verification methods even for highly experienced operators. Additionally, repeat radiofrequency procedures that are performed when nerves regenerate, often a year or more later, are often less effective and more difficult because the nerves' distribution after regeneration creates additional anatomic variability.

### SUMMARY

Features and advantages of the invention will be more readily understood from the following detailed description. In one aspect, a biomarker detection system is disclosed that includes a target biomarker in a biological system. The disclosed biomarker detection system includes real-time fluorescent probe. The disclosed probe for real-time sensing of a target biomarker includes a needle having an opening, a fluorescent probe encased within the needle. The fluorescent probe includes a fluorescent coating disposed within and covering the opening of the needle, wherein the fluorescent coating is in contact with biological tissue. In some embodiments, the biological tissue includes blood. The disclosed fluorescent probe also includes an ion-consuming coating within the needle and adjacent to the fluorescent coating and a fiber optic waveguide in contact with the ion-consuming coating.

In this disclosure, the terms:

“optical receiver” refers to a light detecting device structured and configured to detect light returning along the optical path from the bioluminescent device to the optical detector;

“optical detector” refers to a device that senses and may measure the amount of light in its optical path;

“optical coupler” refers to a device that is structured and configured to couple light between a fluid channel and at least one optical fiber; and

“optical filter” refers to a device that receives light and allows only light with specific properties such as wavelength, polarity, intensity, or other selective properties to pass therethrough.

In view of these considerations, it would be desirable to provide systems and methods that provide real-time feedback to assist in the precise placement of surgical instruments into patients' anatomies.

### BRIEF DESCRIPTION OF THE DRAWINGS

The following description should be read with reference to the drawings. The drawings, which are not necessarily to scale, depict examples and are not intended to limit the scope of the disclosure. The disclosure may be more completely understood in consideration of the following description with respect to various examples in connection with the accompanying drawings, in which:

FIG. 1 is a schematic quasi-cross-sectional view of an illustrative example of a portion of a biomarker detection system according to the present disclosure;

FIG. 2 is a schematic quasi-cross-sectional view of another illustrative example of a portion of a biomarker detection system according to the present disclosure;

FIG. 3A is a schematic cross-sectional view of still another illustrative example of components of a portion of a biomarker detection system according to the present disclosure;

FIG. 3B is a schematic cross-sectional view that provides an enlarged depiction illustrating details of some of the components of the system of FIG. 3A;

FIG. 4 is a schematic cross-sectional view of yet another illustrative example of components of portion of a biomarker detection system according to the present disclosure;

FIG. 5A is a schematic cross-sectional view of still yet another illustrative example of a portion of a biomarker detection system according to the present disclosure;

FIG. 5B is a schematic cross-sectional view that provides an enlarged depiction illustrating details of some of the components of the system of FIG. 5A;

FIG. 6A is a schematic cross-sectional view of yet still another illustrative example of a portion of a biomarker detection system according to the present disclosure;

FIG. 6B is a schematic cross-sectional view that provides an enlarged depiction illustrating details of some of the components of the system of FIG. 6A;

FIG. 7A is a schematic cross-sectional view of an embodiment of a biomarker detection needle according to the present disclosure;

FIG. 7B is a schematic plan view of the needle of 7A from a viewpoint at the left of the needle as depicted in FIG. 7A;

FIGS. 8A, 8B, 8C, and 8D are schematic cross-sectional views down the bores of needles according to the present disclosure that provide optical fibers and lumens along their lengths;

FIG. 9 is a schematic cross-sectional diagram of an embodiment of a fiber optic sensor for distinguishing between air and liquid useful in a provided disclosure;

FIG. 10 is a schematic cross-sectional view down the bore of an illustrative example of a needle system that can incorporate a disclosed fiber optic air sensor;

FIG. 11 is a schematic cross-sectional view of an embodiment of a needle system that can incorporate a sonic air sensor;

FIG. 12A is a schematic plan view of an illustrative example of an embodiment of a needle system that can incorporate an electrical air sensor;

FIG. 12B is a schematic side cross-sectional view of the needle system of FIG. 12A;

FIG. 12C is a schematic cross-sectional view down the bore of the needle system of FIG. 12A;

FIG. 13A is a schematic side cross-sectional view of an illustrative example of another embodiment of a needle system that can incorporate an electrical air sensor;

FIG. 13B is a schematic cross-sectional view down the bore of one configuration of the needle system of FIG. 13A;

FIG. 13C is a schematic cross-sectional view down the bore of another configuration of the needle system of FIG. 13A;

FIG. 14 is a schematic Jablonski diagram showing possible transitions between electronic states of a molecule;

FIG. 15 is a chemical structure of fluorescein; and

FIG. 16 is planar side cross-section illustration of an embodiment of a real-time fluorescent probe.

The disclosed biomarker detection system and real-time fluorescent probe can improve upon some of the shortcomings in the present art. Its use can improve surgical outcomes and cost structure, particularly with spinal and other minimally invasive surgical procedures. The disclosed biomarker detection device can take the operator dependency out of finding target biomarker materials instead of relying on tactile feel, contrast agents, anatomical landmark palpitation, and visualization under image-guided modalities thereby improving the safety and efficacy of procedures requiring biomarker identification.

### DETAILED DESCRIPTION

The present disclosure relates to systems and methods used to detect biological substances, such as bodily fluids and tissues, including blood, and for distinguishing between bodily media, such as liquid and air. Various embodiments of systems and methods are to be described in detail with reference to the drawings, wherein like reference numerals may represent like parts and assemblies throughout the several views. Reference to various embodiments does not limit the scope of the systems and methods disclosed herein. Additionally, any examples set forth in this specification are not intended to be limiting and merely set forth some of the many possible embodiments for the systems and methods. It is understood that various omissions and substitutions of equivalents are contemplated as circumstances may suggest or render expedient, but these are intended to cover applications or embodiments without departing from the spirit or scope of the disclosure. Also, it is to be understood that the phraseology and terminology used herein are for the purpose of description and should not be regarded as limiting.

The present disclosure provides systems and methods structured, configured, and/or capable of detecting one or more biomarkers via the interaction(s) of the biomarkers with one or more detection materials, and the optical detection of said interaction(s). In some examples, the interaction of a biomarker with a detection material can result in a luminescent emission of light that can be sensed, with said sensing of luminescent light providing evidence of the interaction, and hence, the presence of the biomarker. In some of these examples, emission of light can be an intrinsic chemiluminescent product of the interaction between the biomarker and the detection material. In some others of these examples, illumination by an external light source of the detection material, when in the presence of the biomarker, can result in a fluorescent or phosphorescent emission of light that can be sensed. The present disclosure provides systems and methods that can provide detection of biomarkers via sensing of chemiluminescence, fluorescence, and/or phosphorescence.

While multiple examples of biomarker detection systems illustrated and described in the present disclosure include, or can be used in conjunction with, needles and fluid delivery systems, the applications of the disclosed biomarker detection systems and methods are not limited to fluid delivery applications. In the present disclosure, medicinal fluids can be delivered through the disclosed fluid delivery systems. Fluid delivery systems incorporating detection technologies of the present disclosure can be employed to deliver wires/leads, nanoparticles, and any suitable pharmacological or otherwise therapeutic agents, including regenerative medicines and chemotherapy drugs.

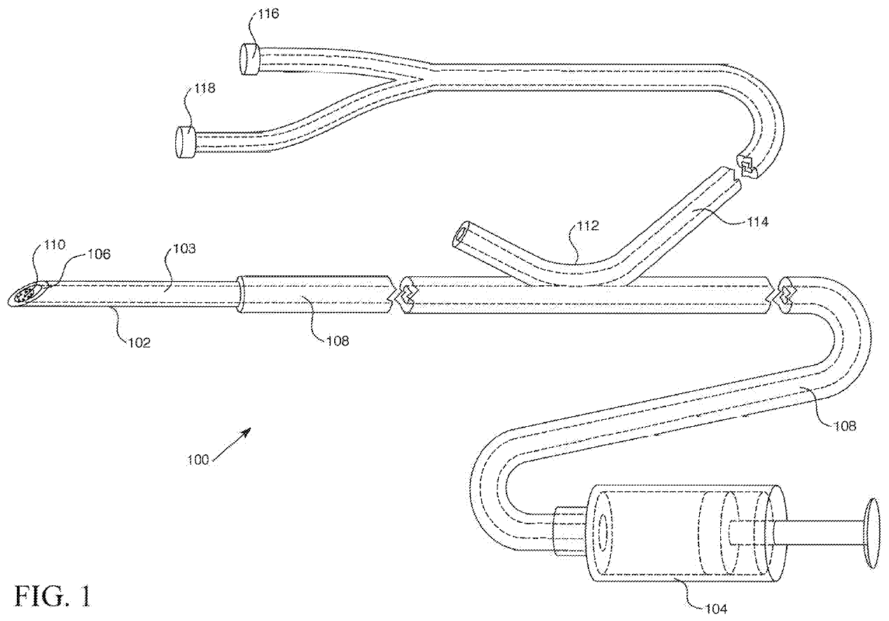

FIG. 1 schematically depicts an illustrative example of a biomarker detection system 100 of the present disclosure. System 100 can include needle 102 having lumen 103 that can deliver a fluid from syringe 104, or other suitable fluid dispensing system, to tip 106 of the needle via fluid channel 108 (said fluid channel including lumen 103). Biomarker luminescent material 110 can be placed at tip 106 or inside and adjacent to tip 106 of needle 102, such as via a coating process.

System 100 can include optical coupler 112 that can be structured and configured to couple light between fluid channel 108 and optical fiber 114. Optical coupler 112 and any other optical couplers of the present disclosure can be structured and configured to perform as a wavelength division multiplexer, which can improve signal-to-noise ratio by, for example, filtering the spectrum of light that reaches a detector. Optical fiber 114 can be selected with core material having an index of refraction that is substantially close or essentially equal to an index of refraction of a fluid in fluid channel 108, such that light can readily be coupled between the two while minimizing reflectance losses and other optical issues that can arise from an optical mismatch. Alternatively, or in addition, the index of refraction of the fluid in channel 108 can be adjusted to substantially or essentially exactly match the index of the core of optical fiber 114, for example, by selecting the concentration of various components of the fluid, such as glucose, alcohol, saccharide salts, and/or any other biocompatible fluid(s). Fluid channel 108 can be structured and configured such that it, with fluid present, can serve as an optical waveguide.

A light source 116 such as a diode laser or other suitable light source can be optically coupled to optical fiber 114 such that light from the light source can be delivered to tip 106 of needle 102 via the optical fiber and the optical wave guide provided by fluid channel 108, and more specifically, to biomarker luminescent material 110 at said tip. Light that is emitted or otherwise scatters from biomarker luminescent material 110 can be returned by the optical path provided by fluid channel 108 and optical fiber 114 to an optical receiver 118, which can be a photo detector or any other suitable light detecting device structured and configured to detect light returning along the optical path from tip 106 of needle 102. Optical coupler 112 can be structured, configured, and tuned such that it can effectively couple light between fluid channel 108 and optical fiber 114 for any relevant optical frequencies, including one or more emission frequencies of light source 116 and the frequency(ies) of luminescence of biomarker luminescent material 110.

Via various mechanisms, light from light source 116 can undesirably reach optical receiver 118, such as via back-reflections from tissue and other back-scattering avenues, adding to the noise read by receiver 118 as it measures the signal of luminescent emission from biomarker luminescent material 110. This source of measurement noise can be countered in multiple ways. As aforementioned, optical coupler 112 can be structured and configured to perform as a wavelength division multiplexer, which can selectively filter the frequencies of light incident upon optical receiver 118 to the frequency(ies) emitted from biomarker luminescent material 110. Alternatively or in addition, optical receiver 118 can include a filter to selectively prevent frequencies other than the frequency(ies) of luminescence of biomarker luminescent material 110 from reaching the optical receiver, such as illumination light from light source 116, and other light that may exist in the environment, such as ambient room lighting. Other measures to improve signal-to-noise can be taken, such as filtering room lighting to attenuate emission at the frequencies of sensitivity of optical receiver 118. Such wavelength and frequency filtration/sensitivity considerations may apply to any relevant systems of the present disclosure.

Another technique that can be employed to improve signal-to-noise for detection at optical receiver 118 of light emitted from biomarker luminescent material 110 is time division multiplexing. By temporally separating the illumination of biomarker luminescent material 110 by light source 116 from the detection of luminescent emission from the material, this source of noise can be circumvented. In an illustrative example, light source 116 can be driven with a fully-on, fully-off square wave. With a light source 116 that can be switched-off sufficiently quickly (that is, fast compared to the decay time constant for luminescent emission from biomarker luminescent material 110), data collection from optical receiver 118 can be gated to be performed only when light source 116 is off, such that no back-reflected/scattered light originating from light source 116 is recorded at optical receiver 118. Potentially, other sources of light (as might be employed in a surgical suite, for example) that might undesirably reach optical receiver 118 could also be driven with the same waveform as light source 116, to prevent their detection. With a sufficiently high frequency, such pulsing could be imperceptible to the human eye. These time division multiplexing methods may be advantageously employed with any compatible systems and methods of the present disclosure.

In an example method of use of system 100, needle 102 can be advanced into a patient by a clinician, with light source 116 activated to provide illumination of biomarker luminescent material 110. A fluid having an optically suitable index of refraction can be present in fluid channel 108 (including lumen 103). Needle 102 can be advanced until tip 106 of the needle encounters a target biomaterial (for example, blood, although other target biomaterials are possible), upon which interaction of the target biomaterial (e.g., blood) with biomarker luminescent material 110, in combination with illumination light from light source 116, can result in emission of light from the biomarker luminescent material, which can be detected by optical receiver 118. A notification system (not illustrated) operatively coupled to optical receiver 118 can inform the clinician that the target biomarker has been detected. The clinician can then position tip 106 of needle 102 in accordance with the detection of the target biomaterial and knowledge of the patient's anatomy (for example, further advancing, stopping advancing, or retracting the needle). With tip 106 of needle 102 appropriately placed, delivery of a therapeutic fluid from the fluid delivery system through fluid channel 108 can be performed.

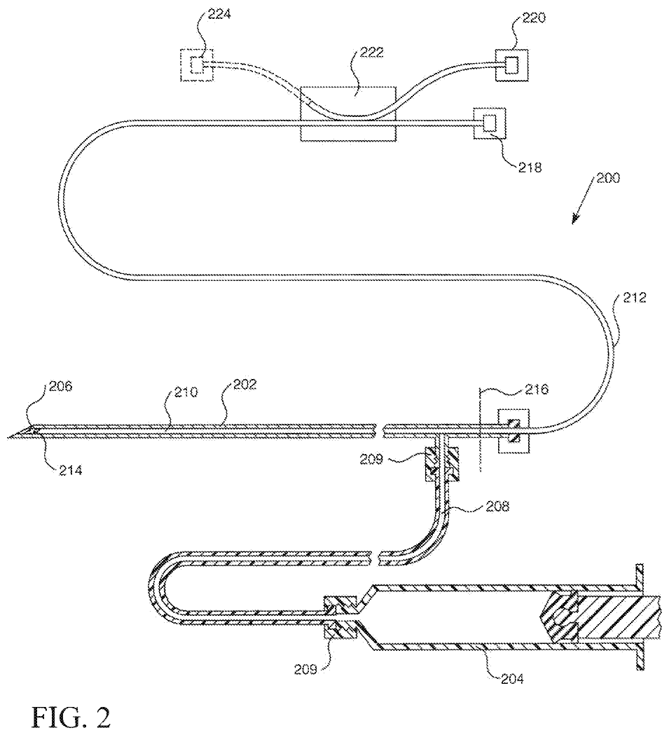

FIG. 2 schematically depicts another illustrative example of another biomarker detection system 200 of the present disclosure. System 200 can include needle 202 that can deliver a fluid from syringe 204, or other suitable fluid dispensing system, to tip 206 of the needle via fluid channel 208. Needle 202 and syringe 204 can be fluidically coupled via one or more fluid connectors 209, which can be any suitable connectors, such as (but not limited to) LUER lock fittings. Fluid channel 208 can include lumen 210 of needle 202. In a configuration of system 200 illustrated in FIG. 2, lumen 210 of needle 202 can be at least partially occupied by optical fiber 212. Optical fiber 212 can include biomarker luminescent material 214 affixed at its distal tip, such as via a coating process. As illustrated in FIG. 2, optical fiber 212 having biomarker luminescent material 214 affixed at its distal tip can be positioned in lumen 210 of needle 202 such that the biomarker luminescent material is located at or near tip 206 of needle 202. In some embodiments of such a configuration, optical fiber 212 can substantially block lumen 210 of needle 202 to the passage of fluid. In some other embodiments, an optical fiber present in lumen 210 may allow at least partial fluid passage. In system 200, optical fiber 212 can be selectively retracted within lumen 210, such that the distal tip of the fiber can be located at or near a location such as location 216, where it may substantially not obstruct flow of fluid from syringe 204 to tip 206 of needle 202 via lumen 210.

System 200 can include light source 218, such as a diode laser or other suitable light source, that can be optically coupled to optical fiber 212 such that light from the light source can be delivered to the distal tip of the optical fiber, and more specifically, to biomarker luminescent material 214 at said tip. Light that is emitted or otherwise scatters from biomarker luminescent material 214 can be returned by optical fiber 212 to optical receiver 220, which can be a photo detector or any other suitable light detecting device structured and configured to detect light returned by optical fiber 212 from the distal tip of the fiber.

System 200 can include optical coupler 222 that can be structured and configured to pass light from light source 218 to biomarker luminescent material 214 at the distal tip of optical fiber 212, and to transmit light emitted from biomarker luminescent material 214 to optical receiver 220. Optical coupler 222 can be tuned, for example as a wavelength division multiplexer, to selectively maximize transmission of light emitted from biomarker luminescent material 214 to optical receiver 220, and to minimize the transmission of such emitted light back toward light source 218. Second optical receiver 224, which can be a photo detector or any other suitable light-detecting device, can be coupled to the optical system of system 200 via fiber optic splitter 222. Second optical receiver 224 can be used to sense the drive signal level of emission from light source 218, which can be used to create a differential signal for purposes of compensating for variations in intensity of light source 218 (for example, drifts due to temperature variations), when interpreting signals at optical receiver 220. This arrangement might also be used, in some cases, to implement phase sensitive detection of the signal received at optical receiver 220 from biomarker luminescent material 214.

System 200 can be used similarly in many aspects as described for system 100. In operation, needle 202 of system 200 can be advanced into a patient with optical fiber 212 positioned in lumen 210 such that biomarker luminescent material 214 at the distal tip of the fiber is disposed at or near tip 206 of needle 202. Once biomarker luminescent material 214 contacts the target biomaterial (e.g., blood), light resulting from such contact can be transmitted up the fiber to optical receiver 220 and detection indicated to a user of the system. Once the needle is properly positioned (e.g., in a bloodstream), optical fiber 212 can be retracted (for example, to 216), opening lumen 210 for the flow of fluid from syringe 204 to delivery at tip 206 of needle 202.

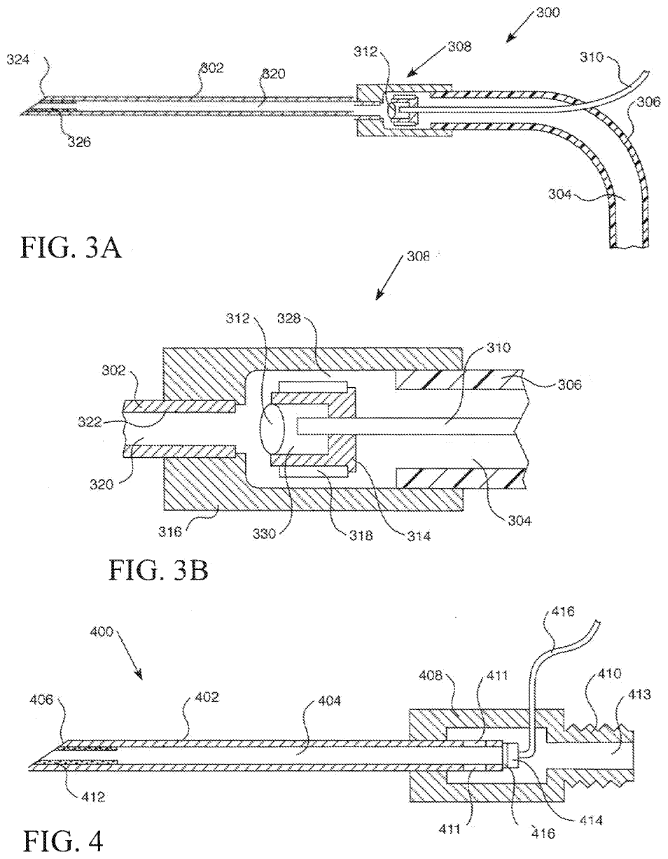

FIG. 3A schematically depicts another illustrative example of components of another biomarker detection system 300 of the present disclosure, and FIG. 3B provides an enlarged depiction that illustrates details of some of the components illustrated in FIG. 3A. System 300 can be used similarly in many aspects as described for system 100 for biomarker detection and therapeutic delivery. The system of FIGS. 3A, 3B can include needle 302 that can deliver a fluid from fluid delivery system (not shown in its entirety) via fluid channel 304 of fluid line 306. System 300 of FIGS. 3A, 3B can include coupler 308 that can couple needle 302 with the fluid delivery system and an optical system (not shown in its entirety). System 300 of FIGS. 3A, 3B, including coupler 308, can employ any suitable fluid connectors (not necessarily illustrated), such as (but not limited to) LUER lock fittings. The optical system can include optical fiber 310 and coupling optics 312, which include a lens or lenses, held in optical alignment by optical mount 314. Optical mount 314 can be held in positional relationship with respect to coupler housing 316 by support web 318. The depicted physical arrangement of and between optical mount 314, coupler housing 316, and support web 318 is merely an example and should not be considered limiting.

Coupling optics 312, when appropriately positioned and aligned with respect to optical fiber 310, can couple or focus illumination light emitting from the optical fiber into lumen 320 of needle 302. Interior walls 322 surrounding lumen 320 of needle 302 can be polished or otherwise smoothed, such as by an electrochemical or other suitable process, such that they can serve as a waveguide for the illumination light so coupled. In some examples, interior walls 322 surrounding lumen 320 of needle 302 can be coated or lined with a thin layer of a dielectric material such as a glass or a polymer having an index of refraction lower than the index of the fluid within the lumen, such that a total internal reflection waveguide similar to an optical fiber results, with a higher-index fluid in the lumen serving as the “core” and the lower-index thin dielectric layer coating the walls of the lumen serving as the “cladding.” (This waveguide configuration can potentially be employed in any system of the present disclosure in which light propagates through a fluid channel, including in needles 102, 402, 502, and 602 of systems 100, 400, 500, and 600, respectively). Illumination light, provided by any suitable light source (not illustrated) such as a diode laser, can be delivered via propagation down lumen 320 of needle 302 to its tip 324, where biomarker luminescent material 326 can be located, such as via a coating process. Light that is emitted or otherwise scatters from biomarker luminescent material 326 can be returned by a reverse optical path (the waveguide of needle lumen 320, then coupled by optics 312 to optical fiber 310) to an optical receiver (not illustrated), which can be a photo detector or any other suitable light detecting device structured and configured to detect light returning by optical fiber 310 from tip 324 of needle 302.

Coupler 308 can include one or more fluid channels 328 that can fluidically communicate between fluid channel 304 of fluid line 306 and lumen 320 of needle 302. Optical mount 314 can define or provide void 330 that can be fluidically sealed from the fluidic path (304, 328, 320) of the fluid delivery system, and in which a vacuum or gas atmosphere of stable refractive index can be maintained, such that effects of index-of-refraction variations on optical coupling can be reduced or eliminated. For the same reason, the fluid-facing side of lens 312 can be a planar surface.

FIG. 4 schematically depicts another illustrative example of components of another biomarker detection system 400 of the present disclosure. System 400 of FIG. 4 can include needle 402 that can deliver a fluid from fluid delivery system (not illustrated) through lumen 404 to tip 406 of needle 402. System 400 of FIG. 4 can include fluid coupler 408 that can couple needle 402 with the fluid delivery system via a fluid connector 410, which can be any suitable fluid connector, such as (but not limited to) a LUER lock fitting. Coupler 408 and/or needle 402 can include or define one or more fluid channels 411 that can fluidically communicate between connector-side fluid channel 413 and lumen 404 of needle 402.

Biomarker luminescent material 412 can be located at tip 406 of needle 402, such as via a coating process. The interior walls surrounding lumen 404 of needle 402 can be polished or otherwise smoothed, such as by an electrochemical or other suitable process, such that they can form a waveguide to efficiently transport light emitted from biomarker luminescent material 412 to optical receiver 414, which can be a photo detector or any other suitable light detecting device structured and configured to detect light emitted from the biomarker luminescent material. A wavelength-discriminating optical filter 416 that is tuned for one or more wavelengths of light emitted by biomarker luminescent material 412 can help reject stray light and improve signal-to-noise. Optical receiver 414 can be electrically connected to detection electronics via connection 418

System 400 of FIG. 4 can be suited for use with biomarker luminescent material 412 that can produce light upon contact with a target biomaterial (e.g., blood) without illumination by an external light source. The omission of an illumination light source can make for a relatively simple biomarker detection device and system. One example of a biomarker luminescent material that does not necessarily require external illumination, which may be used with the system of FIG. 4, is luminol, which is discussed further elsewhere herein.

Aside from the omission of illumination of biomarker luminescent material 412 by an external light source, system 400 can be used similarly in many aspects as described for system 100 for biomarker detection and therapeutic delivery.

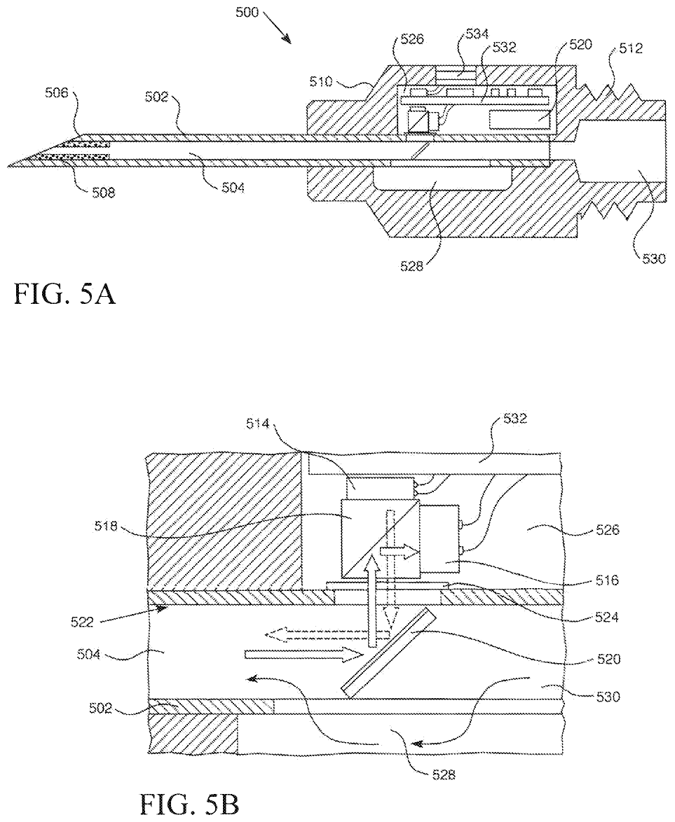

FIG. 5A schematically depicts an illustrative example of a “self-contained” biomarker detection needle system 500 of the present disclosure, and FIG. 5B provides an enlarged depiction that illustrates details of some of the components of system 500. System 500 can include needle 502 having lumen 504 and tip 506, at which biomarker luminescent material 508 can be located, such as via a coating process. System 500 can include mount 510 to which needle 502 can be mounted and connected to a fluid delivery system (not illustrated) via a fluid connector 512, which can be any suitable fluid connector, such as (but not limited to) a LUER lock fitting.

Mount 510 can house optics and electronics to enable bio detection with biomarker luminescent material 508. At mount 510, system 500 can include light source 514 such as a diode laser or other suitable light source, and optical receiver 516, which can be a photo detector or any other suitable light detecting device. The optical system of mount 510 can include beam splitter 518 and mirror 520. With such an optical arrangement, illumination light from light source 514, represented by broken-outline hollow arrows in FIG. 5B, can be directed down lumen 504 of needle 502 toward the needle's tip 506, where it can illuminate biomarker luminescent material 508. Light that is emitted or otherwise scatters from biomarker luminescent material 508 can be returned by a reverse optical path, as represented by solid-outline hollow arrows, to optical receiver 516. Interior walls 522 surrounding lumen 504 of needle 502 can be polished or otherwise smoothed, such as by an electrochemical or other suitable process, such that they can serve as a waveguide for light propagating in needle 502. The optical system of mount 510 can also include optical window 524 that can provide a barrier for fluids in lumen 504 of needle 502 from entering optical/electronic space 526 of mount 510. Optical window 524 can be manufactured to selectively filter wavelengths (e.g., selectively passing illumination light from light source 514 and light emitted from biomarker luminescent material 508, while blocking undesired wavelengths). Selective filtering can also be implemented at one or more surfaces of beam splitter 518.

Mount 510 and/or needle 502 can include or define one or more fluid channels 528 that can fluidically communicate between a connector-side fluid channel 530 and lumen 504 of needle 502, providing a fluid bypass around mirror 520.

Light source 514 and optical receiver 516 can be electronically coupled to circuit board 532, which can provide power and control signals to the devices and receive and process signals or other information from the devices. In FIG. 5B, both light source 514 and optical receiver 516 are illustrated as being electronically coupled to circuit board 532 via wire bonding, but this is not limiting and any suitable functional connections (such as surface mount technology) between the devices and the circuit board can be employed. The term “circuit board” as used in relation to element 532 of system 500 is used generically and should not be considered to be limiting. Circuit board 532 can include a printed circuit board with multiple discrete components, a single chip processor or “system-on-a-chip,” multiple sub-boards, a hybrid system, or any other suitable arrangement capable of powering and carrying out biomarker detection system functionality with the elements of system 500. Mount 510 can host or support any suitable user interface elements 534 which can include (but are not limited to) buttons, visual indicators such as light-emitting diodes, and audio annunciators/speakers. Circuit board 532 can include one or more wireless interfaces, such as a BLUETOOTH interface, which can incorporate any BLUETOOTH features necessary or desirable for operation, such as a detection signal, self-test, battery status, and so on. Mount 510 can house energy storage device 522 which can be a battery or any other suitable device, which can provide operational power for light source 514, optical receiver 516, circuit board 532 and other appropriate components hosted by mount 510. System 500 can be used similarly in many aspects as described for system 100 for biomarker detection and therapeutic delivery.

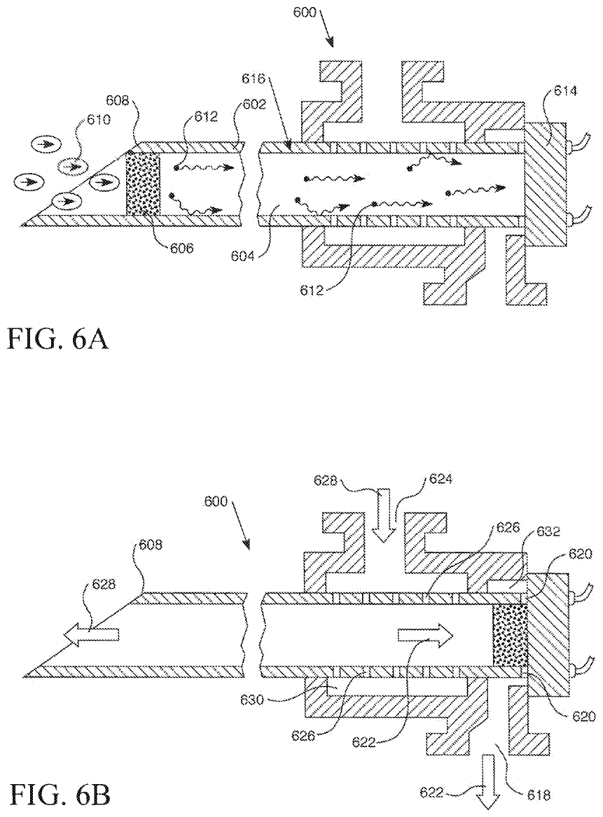

FIGS. 6A and 6B schematically depict another illustrative example of components of another biomarker detection system 600 of the present disclosure. FIG. 6A generally depicts a detection configuration of system 600 and FIG. 6B generally depicts a post-detection fluid-delivery configuration. System 600 can include needle 602 having lumen 604. In the configuration of FIG. 6A, semi-permeable barrier 606 disposed at or near tip 608 of needle 602 can prevent a biomarker luminescent material fluid present in lumen 604 from exiting the needle at its tip, and potentially entering a patient's body. The use of semi-permeable barrier 606 in system 600 can enable the provision of a large reservoir of biomarker luminescent material fluid in lumen 604 of needle 602, providing for greater illumination and extended duration of sensitivity as compared with other arrangements lacking such a reservoir.

Semi-permeable barrier 606 can be at least semi-permeable to target biomaterial 610, such that the target biomaterial can come into contact with, and react with, the biomarker luminescent material fluid present in lumen 604. In some examples, semi-permeable barrier 606 can be structured and configured such that it selectively allows passage of iron ions or iron-containing compounds, as found, for example, as a component of blood. The biomarker luminescent material fluid present in lumen 604 can be a material that is reactive with such iron ions or iron-containing compounds. In some embodiments, the blood stays active indefinitely. Once iron in any form present in blood contacts the biomarker luminescent material and emits radiation, the iron is typically not consumed in the reaction. In some embodiments, the target biomaterial and biomarker luminescent material fluid can react such that photons 612 are generated by said reaction. As illustrated, such photons 612 could be generated in a variety of locations within lumen 604, depending on the penetration of the target biomaterial past barrier 606 and into the volume of the lumen. In some embodiments, semi-permeable barrier 606 can be translucent or at least partially transparent, to allow photons 612 generated by reactions within the barrier to exit the barrier for detection.

Photons 612 produced as a result of contact between target biomaterial 610 and biomarker luminescent material fluid can propagate within lumen 604 of needle 602 to optical receiver 614, which can be a photodetector, or any other suitable light-detecting device structured and configured to detect such light. Interior walls 616 surrounding lumen 604 of needle 602 can be polished or otherwise smoothed, such as by an electrochemical or other suitable process, such that they can serve as a waveguide for light propagating in the needle.

Where in FIG. 6A, biomarker detection system 600 is depicted in a detection configuration, in FIG. 6B the system is depicted in fluid-delivery configuration. System 600 can be reconfigured from the detection configuration of FIG. 6A to the fluid-delivery configuration of FIG. 6B following successful detection of a target biomaterial, but this is not limiting, and such a reconfiguration is not necessarily dependent upon successful biomaterial detection.

With reference to FIG. 6B, reconfiguration can be enacted by withdrawal of biomarker luminescent material fluid from lumen 604 of needle 602 via fluid extraction port 618, which can be in fluidic communication with the lumen of the needle via extraction holes 620. Extraction holes 620 can be formed by any suitable process (conventional machining, laser drilling, etching, and so on). Withdrawal of biomarker luminescent material fluid is indicated by arrows 622, which indicate the direction of biomarker luminescent material fluid flow during withdrawal. Semi-permeable barrier 606 can be slidably configured in lumen 604 of needle 602. As biomarker luminescent material fluid is withdrawn from lumen 604, a slidable semi-permeable barrier 606 can be drawn in the proximal direction (toward the right of FIGS. 6A and 6B) from its prior distal position (at tip 608 of needle 602). Semi-permeable barrier 606 is illustrated at a proximal end of lumen 604, after substantially complete withdrawal of biomarker luminescent material fluid from the lumen. In some alternative examples, semi-permeable barrier 606 could take the form of a non-slidable burst-barrier. It may be generally desirable for such a burst-barrier not to generate any particulates upon bursting.

With biomarker luminescent material fluid withdrawn from lumen 604 of needle 602, system 600 can be used for delivery of fluid from a fluid delivery system (not illustrated) via fluid input port 624, which can be in fluidic communication with the lumen of the needle via delivery holes 626. Delivery holes 626 can be formed by any suitable process (conventional machining, laser drilling, etching, and so on). Delivery of a fluid from a fluid delivery system is indicated by arrows 628.

Note that space 630 in the cross-sectional view of FIGS. 6A and 6B can be in fluidic communication with fluid input port 624, for example via an annular space that surrounds needle 602. Similarly, space 632 can be in fluidic communication with fluid extraction port 618.

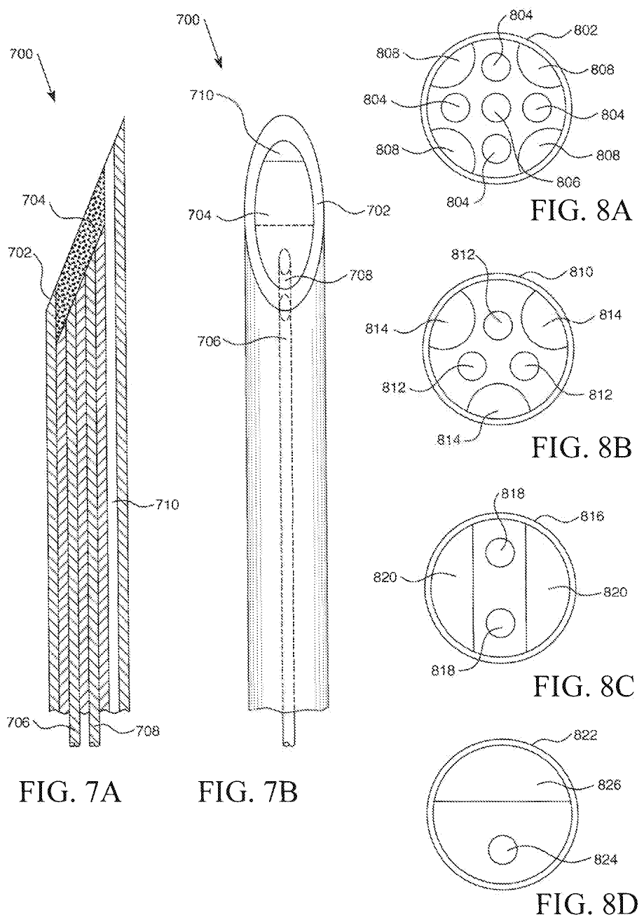

Other needle configurations are possible for biomarker detection via sensing luminescent emissions produced as a result of contact between a biomarker luminescent material and a target biomaterial. FIG. 7A is a schematic cross-sectional view of a biomarker detection needle 700 and FIG. 7B is a schematic plan view of needle 700 from a viewpoint at the left of the needle in FIG. 7A. At its tip 702, needle 700 can include a biomarker luminescent material 704. Needle 700 can include one or more optical fibers 706, 708. One of optical fibers 706, 708 can be used to deliver illumination light from light source (not illustrated), such as a diode laser or other suitable light source, to biomarker luminescent material 704, and the other of the two optical fibers can be used to transport light emitted from the biomarker luminescent material to an optical receiver (not illustrated), which can be a photo detector or any other suitable light detecting device. Needle 700 can include a lumen 710 suitable for fluid delivery from a syringe or other suitable fluid dispensing system (not illustrated).

The configuration of needle employs non-retracting optical fibers 706, 708 for high-efficiency transport of illumination light and light emitted by the biomarker luminescent material, and simultaneously provides a lumen that is always open for fluid delivery to the tip 702 of the needle. In comparison, in system 200 of FIG. 2, retraction of optical fiber 212 from tip 206 to location 216 may be needed to open lumen 210 for fluid delivery. In some other embodiments of the present disclosure, such as system 100 of FIG. 1, the open lumens of needles may be used to provide optical waveguides for light transportation, without an optical fiber or fibers extending to the distal tip of said needles. In many instances, optical fibers can provide higher efficiency transport of light than the lumen of a needle.

FIGS. 8A, 8B, 8C, and 8D are schematic cross-sectional views down the bores of needles that provide optical fibers and lumens along their lengths to the needles' tips, similar to needle 700 of FIGS. 7A and 7B. Needle 802 of FIG. 8A can include five optical fibers, with outer fibers 804 being illumination fibers delivering illumination light from a light source to a biomarker luminescent material and inner fiber 806 being a sensing or detection fiber used to transport light emitted from the biomarker luminescent material to an optical receiver. This arrangement of four outer illumination fibers and one inner detection fiber is just an example and should not be considered limiting. Other fiber arrangements are contemplated. Needle 802 can include one or more lumens 808 suitable for fluid delivery. In some examples, multiple lumens can be employed to provide higher fluid conductance for fluid delivery from a common fluid reservoir. In some other examples, multiple lumens can be employed to provide independent delivery paths for different fluids.

Needle 810 of FIG. 8B can include three optical fibers 812 and three lumens 814 suitable for fluid delivery. Needle 816 of FIG. 8C can include two optical fibers 818 and two lumens 820. Needle 822 of FIG. 8D can include single optical fiber 824 and single lumen 826. These are just examples, and other quantities of optical fibers and lumens can be provided and employed in biomarker detection needles contemplated in the present disclosure. An optical fiber in a needle having a single optical fiber can be employed for both illumination and detection by using, for example, a fiber optic splitter (similar to splitter 222 of system 200 of FIG. 2) for handling coupling of illumination light and detection light with the single fiber. Alternately, an optical fiber in a needle having a single optical fiber can be employed only for transporting detection light from a biomarker luminescent material to an optical receiver in detection arrangements that do not require illumination light.

In some examples contemplated in the present disclosure, instruments with multiple optical fibers, similar to (but not limited to) the needles illustrated in FIGS. 7A, 7B, 8A, 8B, and 8C can be used for devices configured for detection of multiple biomarkers and/or other detectable substances. Each of multiple fibers can be used for independent detection and/or illumination channels. For example, different biomarker luminescent materials can be coated at the ends of different detection fibers at a needle tip, such that different luminescent detection signals can be sensed independently. Different biomarker luminescent materials may have different illumination requirements, which can be provided by multiple illumination fibers. As discussed elsewhere herein, a separate fiber can be employed for air/gas detection.

Biomarker luminescent materials used for biomarker detection in systems and methods of the present disclosure can exploit various different luminescent phenomena. Some biomarker luminescent materials can rely upon chemiluminescence, a chemical reaction that can occur upon contact between a biomarker luminescent material and target biomarker can, without additional energy input, result in light emission that can be sensed as a signal of detection of the biomarker. Systems 400 and 600, which do not necessarily include a light source, may be particularly suited for use with chemiluminescent biomarker luminescent materials, given that they may be less complex (at least in optical complexity) than systems that do include light sources. However, potentially any of systems 100, 200, 300, 400, 500, and 600, and any of needles 700, 802, 810, 816, and 822, could be used in conjunction with chemiluminescent detection, although the inclusion of light sources in some of said systems may be irrelevant to detection of light produced by chemiluminescence.

Some other biomarker luminescent materials can employ photoluminescence (e.g., fluorescence and/or phosphorescence) that is activated by contact between a biomarker luminescent material and target biomaterial. Illumination light for photoluminescence can be provided by light sources of illustrative example systems 100, 200, 300, and 500, and can be transported by optical fibers (and/or in some cases, other waveguides) of systems 100, 200, 300, 500, and at least some of needles 700, 802, 810, 816, and 822.

Some biomarker luminescent materials may exhibit photoluminescence in the absence of a target biomarker, and upon exposure to the target biomarker, the photoluminescence can cease or reduce.

Physical characteristics of biomarker luminescent material coatings can reflect a balance between competing factors. A thin coating can be translucent enough to allow illumination light to penetrate in, and emissions to escape for detection, while a thicker coating can provide greater biomarker detection material and a stronger emission signal. Coatings can include cross-linked hydrophilic coatings. The cross-linked hydrophilic coatings can include the biomarker luminescent material as a part of the coating or can encapsulate or seal it to the delivery device. Porosity of the biomarker luminescent material may be desirable to facilitate interaction between biomarkers and the material.

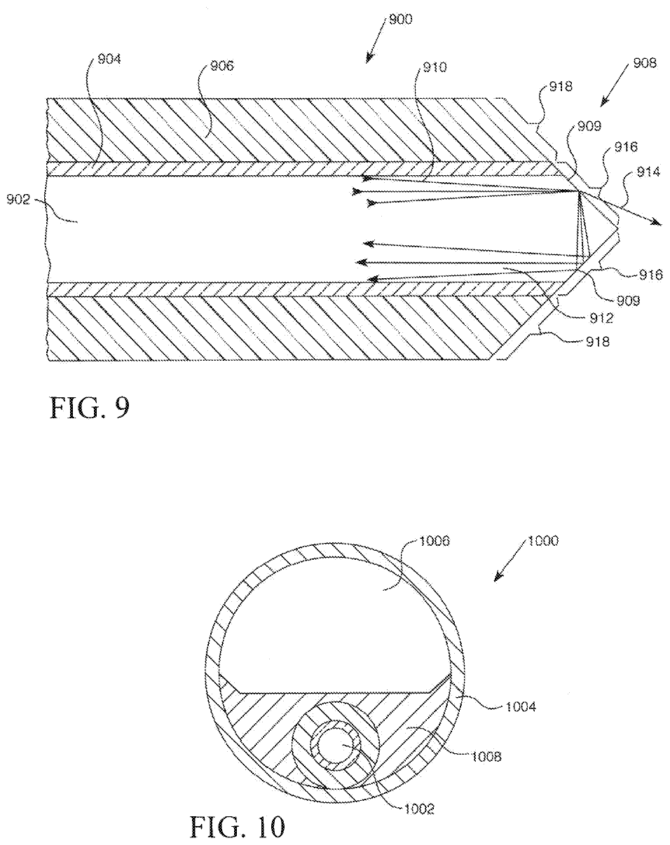

FIG. 9 is a schematic cross-sectional diagram of a fiber optic sensor 900 for distinguishing between air and liquid (which may be referred-to herein as a “fiber optic air sensor”). Sensor 900 can include fiber optic core 902, which can be surrounded by cladding 904, which in turn can be surrounded by buffer 906. Buffer 906 can include one or more buffer layers, such as a primary buffer layer and a secondary buffer layer. Fiber optic air sensor 900 can include any other suitable layers (not illustrated) for strength, protection, etc.

Fiber optic air sensor 900 can be structured and configured to distinguish between liquid and air at a detection end 908 based upon whether light from a light source (not shown) propagating within the fiber toward the detection end (i.e., from left to right in FIG. 9) experiences total internal reflection upon incidence upon faces 909 of fiber core 902 at the detection end. An example bundle of light rays are illustrated as propagating within the core 902 toward (at 910) detection end 908, and then, after reflecting off faces 909, propagating away (at 912) from the detection end. In the case of total internal reflection, rays of light that are incident upon faces 909 at angles of incidence shallower than the critical angle for total internal reflection can be essentially completely reflected. If incident at an angle steeper than the critical angle, then in general a ray can be partially reflected within the core 902 (as at 912) and partially refracted out of the fiber, as illustrated schematically for ray 914.

Faces 909 can be structured to retroreflect rays of light propagating within core 902 toward detection end 908. They can, for example, be oriented at 45 degrees with respect to the longitudinal axis of core 902. In some embodiments, they can be arranged in a cube corner configuration. Faces oriented at 45 degrees with respect to the longitudinal axis (which is essentially the light propagation axis) of core 902 can be suitably oriented for discrimination between air and liquid. For a fused silica fiber, the critical angle for total internal reflection relative to an external medium of air is approximately 43 degrees, and the critical angle for total internal reflection relative to an external medium of water is approximately 67 degrees. Therefore, light propagating along the longitudinal axis of core 902 and incident upon a face 909 that is oriented at 45 degrees with respect to the longitudinal axis can be incident upon the face at shallower than the critical angle for air and steeper than the critical angle for water.

In operation, a detection scheme can include an optical receiver (not illustrated) that can be suitably configured to detect light from the light source that has retroreflected from detection end 908. This retroreflection signal generally can be brighter when faces 909 of detection end 908 are exposed to an external medium of air, resulting in total internal reflection, as opposed to when the faces are exposed to liquid (and hence not resulting in total internal reflection). Faces 909 can include coatings to enhance their detection utility. Inner portions 916 of faces 909, including the portions of the faces where light in the core can be incident, can have a hydrophobic coating, to repel residual liquid on the faces when the detection end 908 is in air. Outer portions 918 of faces 909 can include a hydrophilic coating to draw liquid away from the inner portions 916.

FIG. 10 is a schematic cross-sectional view down the bore of an illustrative example of a needle system 1000 that can incorporate a fiber optic air sensor 1002. Sensor 1002, which can be like fiber optic air sensor 900 of FIG. 9, can be disposed within hypodermic needle 1004 within mold 1008. Hypodermic needle 1004 can enclose fluid channel 1006 for delivery of medicinal fluids, but this is not limiting, and in other embodiments the needle can deliver or house other therapeutic payloads and/or devices. It is contemplated that fiber optic air sensors can be incorporated into other configurations of medical devices, including in combination with biomarker detection systems as described herein.

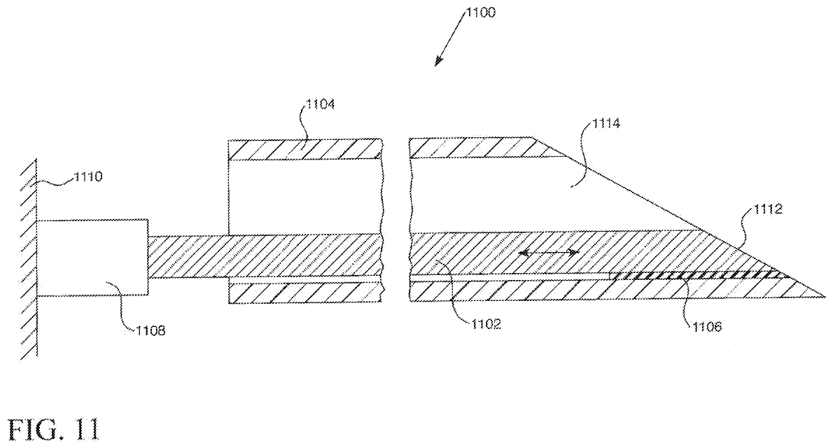

FIG. 11 is a schematic cross-sectional view of needle system 1100 that can incorporate a sonic probe for distinguishing between air and liquid (which may be referred-to herein as a “sonic air sensor”). Sensing rod 1102 can be mounted within hypodermic needle 1104 via one or more elastomeric attachments 1106. Sensing rod 1102 can be driven longitudinally (as indicated by the arrow superimposed thereupon) by piezoelectric actuator 1108 relative to a reaction mass 1110 at an appropriate frequency. Tip 1112 of sensing rod 1102 at the distal end of needle system 1100 can be in mechanical contact with whatever medium may exist at its location, whether tissue, liquid, or gas. Each of these media can present a different mechanical impedance to the motion of sensing rod 1102, with impedance generally decreasing in order (tissue>liquid>gas). Sonic/mechanical impedance can be measured in a variety of ways, including, but not limited to, (a) apply constant drive force and measure amplitude; (b) drive to constant amplitude and measure drive force; and/or (c) measure the phase shift between drive force and motion. Needle system 1100 can enclose a fluid channel 1114 for delivery of medicinal fluids, but this is not limiting, and in other embodiments the needle can deliver or house other therapeutic payloads and/or devices. It is contemplated that sonic air sensors can be incorporated into other configurations of medical devices, including in combination with biomarker detection systems as described herein.

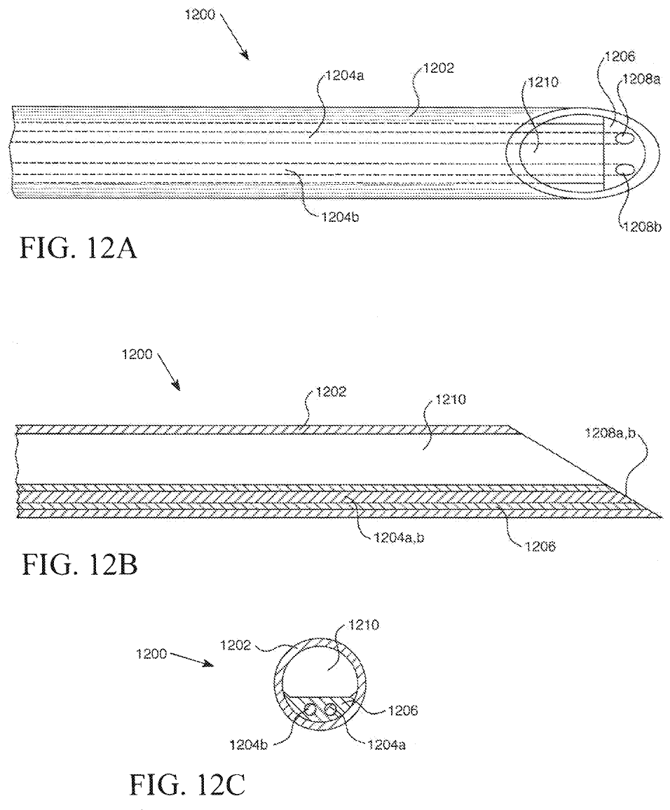

FIGS. 12A, 12B, and 12C are, respectively, a schematic plan view, a schematic side cross-sectional view, and a schematic cross-sectional view down the bore of an illustrative example of a needle system 1200 that can incorporate an electrical sensor for distinguishing between air and liquid (which may be referred-to herein as an “electrical air sensor”). Conductors 1204a, 1204b can be molded in insulator 1206 residing within hypodermic needle 1202. Hypodermic needle 1202 can be grounded and conductors 1204a, 1204b can be connected to an electrical driving and sensing apparatus (not illustrated). The ends of conductors 1204a, 1204b can be polished at the distal tip of needle 1202 such that their faces 1208a, 1208b can be in conductive contact with whatever medium is at the tip of the needle. In general, the electrical conductivity between faces 1208a and 1208b of conductors 1204a, 1204b can depend on said medium. For example, the conductivities [measured in (ohm·cm)⁻¹] of human blood plasma (13.5×10⁻³) is significantly different from that of gastric juice (24×10⁻³) and urine (40×10⁻³). The conductivity of air would generally be significantly lower. A hydrophobic coating can be placed at the end of the needle to aid in rejection of liquid from the faces 1208a and 1208b of conductors 1204a, 1204b upon encountering an air space. By monitoring the conductivity between the faces 1208a and 1208b of conductors 1204a, 1204b, differences in media present at the tip of needle system 1200 can be detected, and in some cases, identified. Needle system 1200 can enclose a fluid channel 1210 for delivery of medicinal fluids, but this is not limiting, and in other embodiments the needle can deliver or house other therapeutic payloads and/or devices. It is contemplated that electrical air sensors can be incorporated into other configurations of medical devices, including in combination with biomarker detection systems as described herein.

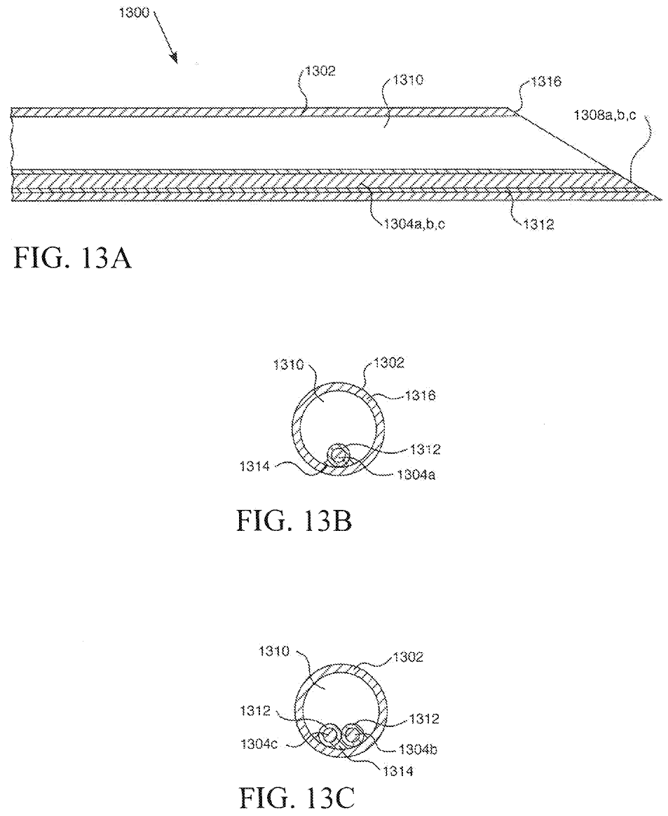

Needle systems similar to system 1200 of FIGS. 12A, 12B, and 12C are illustrated in FIGS. 13A, 13B, and 13C, which are, respectively, a schematic side cross-sectional view of a illustrative example of a needle system 1300 that can incorporate an electrical air sensor, and schematic cross-sectional views down the bores of two alternative configurations of needle system 1300. In the configurations of FIGS. 13A, 13B, and 13C, wires can be bonded within the lumens of hypodermic needle 1302, as compared with needle system 1200, in which conductors 1204a, 1204b are molded within insulator 1206 within the lumen of needle 1202. Wires of the configurations of FIGS. 13A, 13B, and 13C each include a conductor 1304a, 1304b, or 1304c, with each conductor surrounded by insulator 1312. The wires can be bonded within the needle lumen with adhesive 1314.

In the configuration of FIG. 13B, a single wire with conductor 1304a can be bonded within needle 1302. In this configuration, conductor 1304a serves as one conductor, and needle 1302 serves as the other conductor for electrical air sensing. In the configuration of FIG. 13C, two wires having conductors 1304b, 1304c can be bonded within needle 1302 and serve as the two conductors needed to complete the air sensing circuit. A hydrophobic coating can be placed at the end of the needle to aid in rejection of liquid from the faces 1308a, 1308b, 1308c of conductors 1304a, 1304b, 1304c, and the end 1316 of needle 1302, upon encountering an air space. Needle system 1300 can enclose fluid channel 1310 for delivery of medicinal fluids, but this is not limiting, and in other embodiments the needle can deliver or house other therapeutic payloads and/or devices.

In an example method of use of a needle system having an optical, sonic, or electrical air-detection system, such as one of systems 1000, 1100, 1200, or 1300, the needle can be advanced into a patient by a clinician, with the air-detection system activated to provide feedback to the clinician. Upon advancement of the tip of the needle into a media of interest, a notification system (not illustrated) operatively coupled to the air-detection system can inform the clinician that the target media has been detected. The clinician can then position the tip of the needle in accordance with the detection of the target media and knowledge of the patient's anatomy (for example, further advancing, stopping advancing, or retracting the needle). With the tip of the needle appropriately placed, delivery of a therapeutic fluid from the fluid delivery system (or other therapeutic action) can be performed.

The present disclosure further contemplates real-time systems and methods for distinguishing between gas (which may be referred to as “air”) and liquid within patients' anatomies to assist in the precise placement of surgical instruments therein. Such systems can include optical, sonic, and/or electrical detection, and can be based upon differences in optical, sonic, and/or electrical impedance.

The present disclosure also contemplates real-time systems and methods for determining the location of a biomarker detection system that can detect the presence of a target biomarker in real-time. In some embodiments, disclosed biomarker detection system can include a probe that can sense the presence of specific types of bodily tissues. For example, a biomarker detection system according to the present disclosure can include a probe that detects iron and thus the presence of blood when in contact with that type of tissue. The disclosed real-time probe for blood can signal whether or not it is in close proximity to blood—signaling the presence of blood when the probe is in contact with blood and the absence of blood when the probe is no longer in contact with blood. Real-time probes according to this disclosure can be useful, for example, in endoscopic procedures that involve probing inside of the body during surgery or in trying to introduce an anesthetic agent in a tight location such as the spinal cavity as discussed above. Such a probe can operate by detecting a concentration of iron in proximity to the probe.

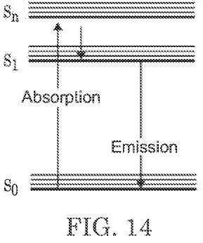

A simplified, typical fluorescence process is shown in the Jablonski diagram illustrated in FIG. 14. Fluorescence is defined as “the molecular absorption of light energy (photon) at one wavelength and its re-emission at another, longer, wavelength.” During that process a photon can be absorbed from, for example, a ground state (S₀) of a fluorescent molecule bumping it up into an excited state such as an excited state (Sₙ) of the energy gap between the ground state and the excited state matches the energy of the absorbed radiation. Frequently, as shown in FIG. 14 the electron can be promoted to an upper excited state such as (Sₙ) in which the excited molecule can lose energy through vibrational and conformation changes until it reaches its lowest excited state (S₁). When the (S₁) state is populated with enough electrons, the excited state of the fluorescent molecule can cascade back to ground state (S₀) emitting a photon of a different wavelength than that absorbed. The emitted photon is almost always of a lower energy (higher wavelength) than the absorbance event. The fluorescence process is cyclical therefore a fluorophore can be excited repeatedly Fluorescence Emission Absorption of a photon and thus excitation to (S1) or (Sn) respectively. Reconversion to (S₀) from (S₁) with emission of radiation (fluorescence).

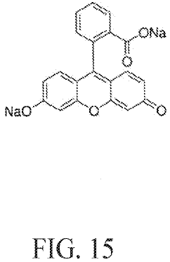

FIG. 15 is a representation of the chemical structure and formula of the molecule, fluorescein. Fluorescein (C₂₀H₁₀Na₂O₅) and closely related structures can persist and remain actively luminescent (fluorescent) for time periods in excess of a typical medical procedure. The needle detection and luminescence with a fluorescein or fluorescein-like) coating can be persistent and can remain actively luminescent for time in excess of the typical medical procedure. Once iron ions (Fe²⁺) from blood contacts the coating, the iron serves as a catalyst for enabling the reemission of absorbed radiation at a different frequency and is not consumed in a reaction.

The fluorescein molecule has a strong affinity and selectivity to iron ions, having two unshared electron pairs available for donation to a metal ion. Once the covalent bond occurs, the molecule gains the ability to fluoresce. This selectivity for chelating iron ions is one of the reasons that Fluorescein is an ideal choice for this sensor. This sensor platform relies upon photoinduced electron transfer (PET) between this fluorophore and its metal-specific chelate, Fe²⁺. Also, in general, the persistence of the fluoresce property is a benefit. In a chemical process where the iron is consumed, such as with luminol, the persistence of the luminescence is limited to the time in which the iron is being consumed, often shorter than a surgical procedure. The intensity of the lamination also decreases over time.

The problem with relatively unlimited florescence time is that when leaving an iron ion rich environment, the sensor remains in the “on” state. If the goal of one embodiment of the sensor is to make a sensor with real-time dynamic response to iron ion concentration, then the key objective is to break these iron ion bonds at a steady rate such that the fluorescence reduces over time as the sensor passes to tissue with lower iron concentration.

The present embodiment designed to measure real-time iron concentration can involve a means to steadily draw the iron ions away from the florescence media coating from the backside of the coating, while the frontside of the florescence media coating is exposed to the sample fluid. The present disclosure introduces a biomarker detection probe that utilizes a second coating underneath the florescence media coating with a stronger affinity for the iron ions. The florescence media coating can be a semi permeable membrane such that the iron ion concentration in this coating creates florescence when the front-side of the coating experiences high iron concentrations and is swept clean of iron ions when the front-side of the coating is exposed to a low iron ion concentration, reducing the intensity of the florescence accordingly. The composition of this second layer can consist of many different materials with the desired properties to clear ions from the detection layer over time.

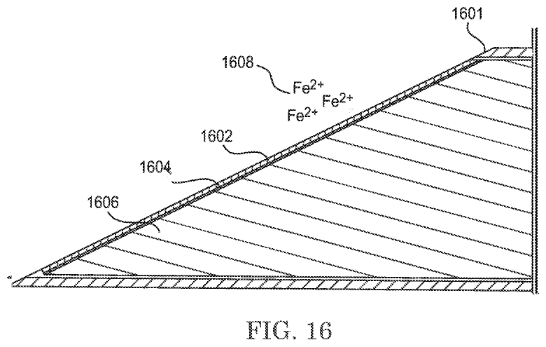

FIG. 16 is planar side cross-section illustration of an embodiment of a real-time fluorescent probe. The real-time fluorescent probe shown in FIG. 16 is incased within the diameter of needle 1601. Needle 1601 has fluorescent coating 1602 (containing, for example, fluorescein or a related molecule) on its outside edge that is in contact with biological tissue when needle 1601 is inserted into a biological system. Fluorescent coating 1602 is disposed upon ion-consuming coating 1604 that effectively removes iron ions after detection. Fluorescent coating 1602 with ion-consuming coating 1604 disposed thereupon is adjacent to fiber optic waveguide 1606 that can conduct radiation emitted from fluorescent coating 1602 to a detector when fluorescent coating 1602 is in the presence of iron ions 1608.

Coating thicknesses and permeability of the different layers can be adjusted to modify the ion sensing response time. Coating compositions must be substantially transparent to the light wavelength emitted by the florescent coating so that emitted light will enter the fiber optic wave guide or selected optical transmission medium.

Another contemplated embodiment can incorporate nanoparticles in the florescence media coating itself wherein the fluorescent media coating can slowly release chemicals to consume the iron ions over time, but not so fast as to inhibit the florescent coating from illuminating in the presence of the target ion. Again, the release rate can be adjusted to modify the ion sensing response time.

What is claimed is:

1. A probe for real-time sensing of a target biomarker comprising:
a needle comprising a tip and having an opening;
a luminescent probe encased within the opening of the needle, wherein the luminescent probe comprises a biomarker luminescent material disposed within and covering the opening of the needle;
a coating comprising a biomarker luminescent material adjacent to or inside the tip of the needle;
wherein the biomarker luminescent material is in contact with biological tissue;
a first ion-consuming coating within the needle and adjacent to the coating.

2. A probe for real-time sensing of a target biomarker according to claim 1, wherein the biomarker luminescent material comprises a fluorescent molecule.

3. A probe for real-time sensing of a target biomarker according to claim 2, wherein the fluorescent molecule comprises fluorescein.

4. A probe for real-time sensing of a target biomarker according to claim 1, wherein the first ion-consuming coating detects iron and the presence of blood.

5. A probe for real-time sensing of a target biomarker according to claim 4, wherein the probe can signal the presence or absence of blood in real-time.

6. A probe for real-time sensing of a target biomarker according to claim 4, further comprising a second ion-consuming coating disposed underneath the first ion-consuming coating wherein the second ion-consuming coating has a stronger affinity for iron than the first ion-consuming coating.

7. A biomarker detection system comprising:
a needle comprising a fluid channel and a tip;
a fluid dispensing system in fluid communication with the needle that can deliver a fluid to the fluid channel;
a biomarker luminescent material at the tip of the needle, or inside and adjacent to the tip of the needle; and
an optical coupler structured and configured to couple light emitted from the biomarker luminescent material between the fluid channel and an optical fiber.

8. A biomarker detection system according to claim 7, wherein the optical coupler can be structured and configured to perform as a wavelength division multiplexer.

9. A biomarker detection system according to claim 7, wherein the optical fiber comprises a core material having an index of refraction substantially equal to an index of refraction of the fluid in the fluid channel.

10. A biomarker detection system according to claim 7, wherein the light emitted from the biomarker luminescent material is in optical communication with a first optical receiver.

11. A biomarker detection system according to claim 10, wherein the first optical receiver comprises a filter to selectively prevent frequencies other than a frequency of light emitted by the biomarker luminescent material from reaching the optical receiver.

12. A biomarker detection system according to claim 10, wherein the first optical receiver utilizes time division multiplexing to improve signal-to-noise for detection at the first optical receiver.

13. A biomarker detection system according to claim 10, further comprising a second optical receiver wherein the second optical receiver comprises a light-detecting device.

14. A biomarker detection system according to claim 13, wherein the second optical receiver is coupled to the light detecting device via a fiber optic splitter.

15. A biomarker detection system according to claim 7, wherein the optical coupler comprises a lens.

16. A biomarker detection system according to claim 7, wherein the optical fiber comprises a fiber optic air sensor.

17. A biomarker detection system according to claim 16, wherein the fiber optic air sensor is disposed within the needle.

18. A biomarker detection system according to claim 7, further comprising a semi-permeable membrane structured and configured to selectively allow passage of iron ions or iron-containing compounds to the biomarker luminescent material.
